(19) United States
(12) Patent Application Publication
(10) Pub. No.: US 2026/0174817 A1
(43) Pub. Date: Jun. 25, 2026

(54) COMPOSITIONS AND METHODS FOR INHIBITION OF PRECANCER CELLS CONTAINING HPV DNA

(71) Applicant: Research Foundation of the City University of New York, New York, NY (US)

(72) Inventor: Linda Saxe Einbond, Tuckahoe, NY (US)

(21) Appl. No.: 19/425,444

(22) Filed: Dec. 18, 2025

Related U.S. Application Data
(60) Provisional application No. 63/736,291, filed on Dec. 19, 2024.
(60) Provisional application No. 63/751,448, filed on Jan. 30, 2025.

(51) Int. Cl. A61K 36/9066; A61K 31/121; A61K 31/343; A61K 31/366; A61K 31/513; A61K 31/731; A61K 36/67; A61K 36/9068; A61P 35/00

(52) U.S. Cl. CPC A61K 36/9066; A61K 31/121; A61K 31/343; A61K 31/366; A61K 31/513; A61K 31/731; A61K 36/67; A61K 36/9068; A61P 35/00

(57) ABSTRACT

Synergistic combinations of compounds that inhibit the growth of cancerous and precancer cells, including cervical cancerous or precancerous cells and/or colon cancerous or precancerous cells.

### CROSS-REFERENCE TO RELATED APPLICATIONS

This application claims priority to, and is a non-provisional of, U.S. Patent Applications 63/736,291 (filed Dec. 19, 2024) and 63/751,448 (filed Jan. 30, 2025), the entirety of which are incorporated herein by reference.

### STATEMENT OF FEDERALLY SPONSORED RESEARCH OR DEVELOPMENT

This invention was made with government support under grant number GM113782 awarded by the National Institute of General Medical Sciences, a part of the National Institute of Health. The government has certain rights in the invention.

### BACKGROUND OF THE INVENTION

Cervical cancer is the fourth most common female cancer worldwide (2020) and is caused by persistent human papillomavirus (HPV) infection. Prophylactic vaccines can prevent common high-risk HPV genotypes that induce cervical cancer. However, these vaccines are not always available in developing countries, and do not always protect against every strain of oncogenic HPV. Moreover, women who are already infected do not benefit from these vaccines. Currently, there is no approved medication to treat cervical precancer or HPV infection. Natural products are attractive since they act on multiple targets, are often readily available, and have a history of use in patients.

Studies have shown that curcumin, tanshinone IIA, ellagic acid, epigallocatechin gallate (EGCG), and the botanicalmixture TriCurin (curcumin: EGCG: resveratrol) inhibit the growth of HPV (+) cervical cancer cells.

The discussion above is merely provided for general background information and is not intended to be used as an aid in determining the scope of the claimed subject matter.

### SUMMARY

Synergistic combinations of compounds that inhibit the growth of cancerous and precancer cells, including cervical cancerous or precancerous cells and/or colon cancerous or precancerous cells. An advantage that may be realized in the practice of some disclosed embodiments is that the combination is synergistically more effective that its component compounds alone.

In a first embodiment, a composition of matter is provided comprising (1) turmeric and tanshinone IIA wherein the composition of matter is free of both epigallocatechin-3-gallate (EGCG) and resveratrol, (2) turmeric and ginger or (3) turmeric and carrageenan.

In a second embodiment, a method of treating cervical cancer or precancer cells in a subject in need thereof is provided, the method comprising: administering to the subject a therapeutically effective amount of a combination of therapeutics selected from the group consisting of (1) turmeric and tanshinone IIA, (2) turmeric and ginger and (3) turmeric and carrageenan, wherein the combination of therapeutics provides a synergistic combination index (CI) of 0.8 or less on HeLa cells.

In a third embodiment, a method of treating colon cancer or precancer cells in a subject in need thereof is provided, the method comprising: administering to the subject a therapeutically effective amount of a combination of therapeutics selected from the group consisting of (1) kava and ginger, (2) dihydroxymethysticin (DHM) and turmeric and (3) dihydroxymethysticin (DHM) and 5-fluorouracil (5-FU), wherein the combination of therapeutics provides a synergistic combination index (CI) of 0.8 or less on HT29 cells.

This brief description of the invention is intended only to provide a brief overview of subject matter disclosed herein according to one or more illustrative embodiments and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1A is a graph showing cell proliferation percentage as a function of changes in concentration of turmeric (90 wt % curcuminoids) and tanshinone IIA concentration.

FIG. 1B is a graph showing cell proliferation percentage as a function of changes in concentration of tanshinone IIA and turmeric (90 wt % curcuminoids).

FIG. 1C is a graph showing cell proliferation percentage as a function of changes in concentration of turmeric (95 wt % curcuminoids) and tanshinone IIA.

FIG. 1D is a graph showing cell proliferation percentage as a function of changes in concentration of tanshinone IIA and turmeric (95 wt % curcuminoids).

FIG. 1E depicts a statistical analysis for the graphs of FIG. 1C and FIG. 1D.

FIG. 2A is a graph showing cell proliferation percentage as a function of changes in concentration of ginger as a function of changes in concentration of turmeric (95 wt % curcuminoids).

FIG. 2B is a graph showing cell proliferation percentage as a function of changes in concentration of turmeric (95 wt % curcuminoids) as a function of changes in concentration of ginger.

FIG. 3A is a graph showing cell proliferation percentage as a function of changes in concentration of carrageenan as a function of changes in concentration of turmeric (95 wt % curcuminoids).

FIG. 3B is a graph showing cell proliferation percentage as a function of changes in concentration of turmeric (95 wt % curcuminoids) as a function of changes in concentration of carrageenan.

FIG. 4A is a graph showing cell proliferation percentage as a function of changes in concentration of ginger as a function of changes in concentration of kava.

FIG. 4B is a graph showing cell proliferation percentage as a function of changes in concentration of kava as a function of changes in concentration of ginger.

FIG. 5A is a graph showing cell proliferation percentage as a function of changes in concentration of turmeric (95 wt % curcuminoids) as a function of changes in concentration of dihydroxymethysticin (DHM).

FIG. 5B is a graph showing cell proliferation percentage as a function of changes in concentration of DHM as a function of changes in concentration of turmeric (95 wt % curcuminoids).

FIG. 6A is a graph showing cell proliferation percentage as a function of changes in concentration of 5-fluorouracil (5-FU) as a function of changes in concentration of DHM.

FIG. 6B is a graph showing cell proliferation percentage as a function of changes in concentration of DHM as a function of changes in concentration of 5-FU.

FIG. 7A is a graph showing cell proliferation percentage of 3T3 feeder cells as a function of changes in concentration of selective compounds.

FIG. 7B is a graph showing cell proliferation percentage of W12 cells (clone 20861, type 2) as a function of changes in concentration of selective compounds.

FIG. 7C is a graph showing cell proliferation percentage of W12 cells (clone 20822, type 1) as a function of changes in concentration of selective compounds.

FIG. 7D is a graph showing cell proliferation percentage of W12 cells (clone 20850, episomal) as a function of changes in concentration of selective compounds.

FIG. 8A is a graph showing cell proliferation percentage of various W12 cells and 3T3 feeder cells as a function of turmeric (95 wt % curcuminoid) concentration.

FIG. 8B is a graph showing cell proliferation percentage of various W12 cells and 3T3 feeder cells as a function of DHM concentration.

FIG. 8C is a graph showing cell proliferation percentage of various W12 cells and 3T3 feeder cells as a function of ginger concentration.

FIG. 8D is a graph showing cell proliferation percentage of various W12 cells and 3T3 feeder cells as a function of tanshinone IIA concentration.

FIG. 9 is a graph showing RT-PRC results indicating tanshinone IIA activates p53.

FIG. 10A depicts a heat map showing binding sites of curcumin and tanshinone IIA to Na⁺/K⁺-ATPase.

FIG. 10B depicts the molecular docking of tanshinone IIA to Na⁺/K⁺-ATPase.

FIG. 10C depicts the molecular docking of curcumin to Na⁺/K⁺-ATPase.

FIG. 10D and FIG. 10E provide two views of the molecular docking of both tanshinone IIA and curcumin to Na⁺/K⁺-ATPase.

### DETAILED DESCRIPTION OF THE INVENTION

This disclosure provides plant-derived compounds for the prevention and treatment of human papillomavirus (HPV)-induced cervical cancer. Specifically, this disclosure provides synergistic combinations of compounds that inhibit the growth of cervical precancer cells, which contain episomal or integrated HPV DNA. In some embodiments, the synergistic combinations are used to treat and/or prevent other HPV-driven cancers such as head and neck cancers, including oropharyngeal cancers. Advantageously, the synergistic combinations permit, in some embodiments, a lower dose to be administered and achieve a therapeutic effect. This lower dose can alleviate toxicity and/or undesirable side effects and delay the development of resistance.

The disclosure demonstrates that specific synergistic combinations of compounds suppress HPV-related cellular processes and serve as therapeutic agents for cervical cancer prevention and treatment. In some embodiments, the combinations are useful for treating colon cancer. The combinations of plant compounds demonstrated in this disclosure play an important role in preventing HPV infection or its progression to cervical cancer. As the combinations inhibit the growth of HPV-related precancerous cells, they may be used as a treatment or in preventative strategies, such as topical treatments or oral supplements, for individuals with HPV infections, especially those at higher risk for developing cervical cancer.

To quantify the interaction between the compounds, the combination index (CI) was determined, using the method of Chou and Talalay (Planta Med. 72, 1200-1206, 2006). This method is a widely recognized method of quantifying drug synergy. CI and its corresponding effects are as follows: >1.3, antagonism; 1.1-1.3, moderate antagonism; 0.9-1.1, additive effect; 0.8-0.9, slight synergism; 0.6-0.8, moderate synergism; <0.6, strong synergism. The CI for combinations of select compounds is shown in Table 1.

TABLE 1

| | | Disease | |
|---|---|---|---|
| Entry | Synergistic combination | condition | CI |
| 1 | tanshinone IIA (2 μM) and turmeric | Cervical cancer | 0.67 |
| | 90 wt % curcuminoids (8 μg/ml) | (HeLa cells) | (Moderate |
| | | | Synergy) |
| | tanshinone IIA (10 μM) and turmeric | Cervical cancer | 0.64 |
| | 90 wt % curcuminoids (2 μg/ml) | (HeLa cells) | (Moderate |
| | | | Synergy) |
| | tanshinone IIA (0.2 μM) and | Cervical cancer | <0.1 |
| | turmeric 95 wt % curcuminoids (0.2 | (HeLa cells) | (Strong |
| | μg/ml) | | synergy) |
| 2 | turmeric 95 wt % curcuminoids (2 | Cervical cancer | 0.53 |
| | μg/mL) and ginger (10 μg/mL) | (HeLa cells) | (Strong |
| | | | synergy) |
| 3 | turmeric 95 wt % curcuminoids (0.1 | Cervical cancer | 0.78 |
| | g/mL) and carrageenan (2 μg/ml) | (HeLa cells) | (Moderate |
| | | | Synergy) |
| 4 | kava (0.8 μg/mL) and ginger (10 | Colon cancer | 0.40 |
| | μg/mL) | (HT29 cells) | (Strong |
| | | | synergy) |
| | kava (2 μg/mL) and ginger (10 | Colon cancer | 0.79 |
| | μg/mL) | (HT29 cells) | (Moderate |
| | | | synergy) |
| 5 | dihydroxymethysticin (DHM) (0.8 | Colon cancer | 0.769 |
| | μg/mL) and turmeric (3 μg/mL) | (HT29 cells) | (Moderate |
| | | | Synergy) |
| | dihydroxymethysticin (DHM) (2 | Colon cancer | 0.788 |
| | μg/mL) and turmeric (1.5 μg/mL) | (HT29 cells) | (Moderate |
| | | | Synergy) |
| | dihydroxymethysticin (DHM) (2 | Colon cancer | 0.563 |
| | μg/mL) and turmeric (3 μg/mL) | (HT29 cells) | (Strong |
| | | | synergy) |
| 6 | dihydroxymethysticin (DHM) (10 | Colon cancer | <0.30 |
| | μg/mL) and 5-fluorouracil (5-FU) (2 | (HT29 cells) | (Strong |
| | μg/mL) | | synergy) |
| | dihydroxymethysticin (DHM) (2 | Colon cancer | 0.56 |
| | μg/mL) and 5-fluorouracil (5-FU) | (HT29 cells) | (Strong |
| | (0.2 μg/mL) | | synergy) |
| | dihydroxymethysticin (DHM) (0.8 | Colon cancer | 0.87 |
| | μg/mL) and 5-fluorouracil (5-FU) | (HT29 cells) | (Moderate |
| | (0.4 μg/mL) | | synergy) |

To assess possible synergistic effects, cells were treated with all combinations of four concentrations of each of the agents tested and a solvent control. Cells were exposed to increasing concentrations of agents for 96 h and the number of viable cells determined by the EZQUANT assay for cervical cancer combinations of (1) tanshinone IIA and turmeric as well as turmeric and ginger.

### Synergistic Combination of Tanshinone IIA Plus Turmeric (90 wt % Curcuminoids)

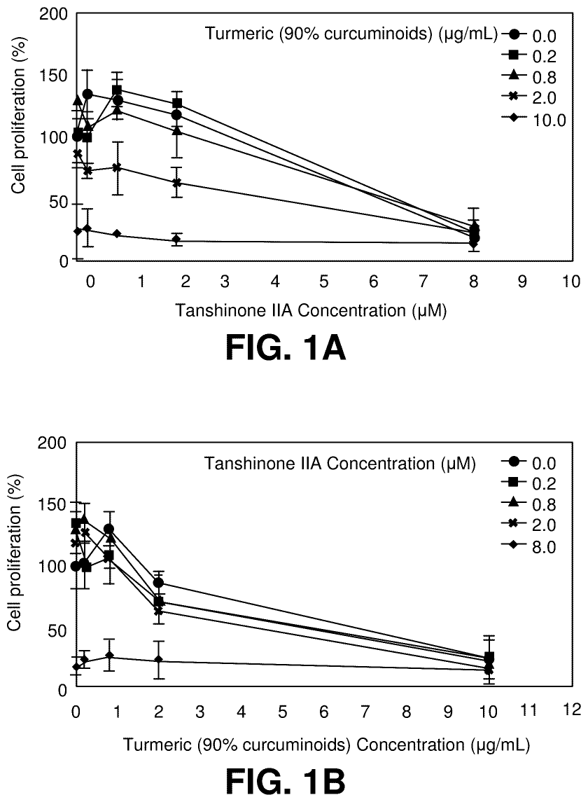

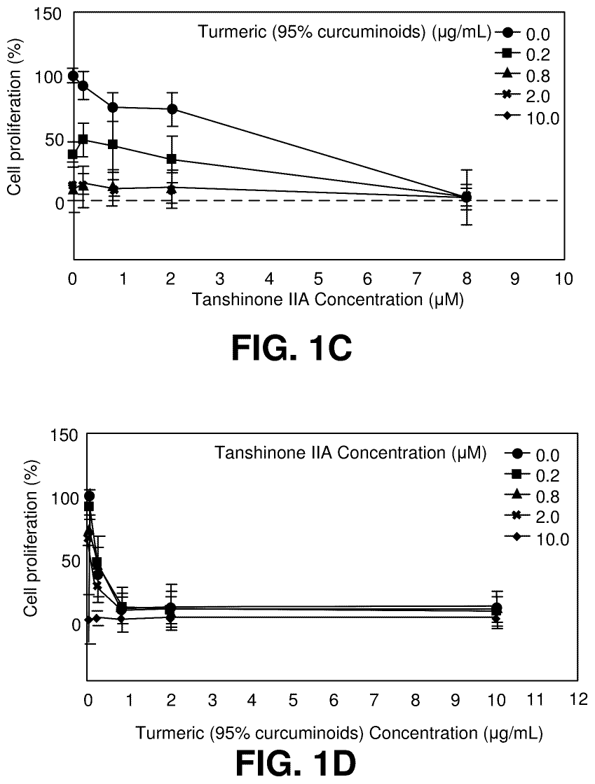

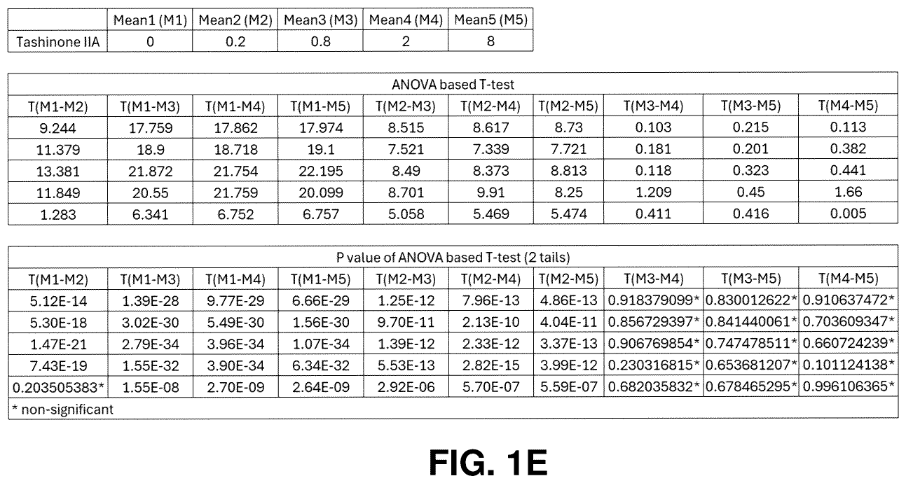

The IC₅₀ values against HeLa cervical cancer cells for tanshinone IIA and turmeric (90 wt % curcuminoids) alone were approximately 6 μM and 6.25 μg/ml, respectively. Increasing concentrations of turmeric were combined with increasing concentrations of tanshinone IIA (FIG. 1A, FIG. 1B). At a dose of 0.2 μM tanshinone IIA, viable cells changed from 134.02% (with tanshinone IIA alone) after 96 hours to 99.23%, 108.26%, 71.65% and 26.54% when combined with turmeric at a dose of 0.2, 0.8, 2, 10 μg/ml, respective (p<0.01). Thus, turmeric enhances the growth inhibitory effect of tanshinone IIA in the human cervical cancer cell line HeLa.

The Combination Index (CI) for the combination of tanshinone IIA 2 μM and turmeric 8 μg/ml was approximately 0.67; while the CI for the combination of tanshinone IIA 10 μM and turmeric 2 μg/ml was approximately 0.64, both indicating moderate synergy.

### Synergistic Combination of Tanshinone IIA Plus Turmeric (95 wt % Curcuminoids)

The IC₅₀ values for tanshinone IIA and turmeric (95 wt % curcuminoids) alone were approximately 4 μM and 0.16 μg/ml, respectively, on HeLa cervical cancer cells. When increasing concentrations of both tanshinone IIA and turmeric (95 wt % curcuminoids) were combined (FIG. 1C, FIG. 1D) a synergistic effect was observed. At a dose of 0.2 μM tanshinone IIA, viable cells changed from 91.96% (with tanshinone IIA alone) to 48.65%, 13.76%, 11.75% and 12.16% when combined with turmeric at a dose of 0.2, 0.8, 2.0 and 10.0 μg/ml, respective (p<0.01). Thus, turmeric enhances the growth inhibitory effect of tanshinone IIA on the human cervical cancer cell line HeLa.

The CI for the combination of tanshinone IIA 0.2 μM and turmeric (95 wt % curcuminoids) 0.2 μg/ml was <0.2, indicating strong synergy. The final percent cell viability was very low indicating the combination is effective to treat cancer. The combination of turmeric (95 wt % curcuminoids) and tanshinone IIA was more effective compared to the combination of turmeric (90 wt % curcuminoids) and tanshinone IIA. A statistical analysis for FIG. 1C and FIG. 1D is provided in FIG. 1E.

Previous studies have found a synergistic effect between tanshinone IIA and TriCurin (a combination of curcuminoids, epigallocatechin-3-gallate (EGCG), and resveratrol). These studies found a CI of 0.84 (at 0.2 μM tanshinone IIA and 0.8 μM TriCurin) for the combination of TriCurin and tanshinone IIA, indicating a slight synergy. At higher concentrations (0.8 μM tanshinone IIA and 1.6 μM TriCurin) the CI was 0.34. Surprisingly, this disclosure demonstrates that a composition that is free of EGCG and resveratrol unexpectedly enhances the synergistic effect against cervical cancer and precancer cells. This is evidenced by a significantly lower CI (CI<0.2) at substantially lower concentrations (e.g., tanshinone IIA 0.2 μM+turmeric 0.2 μg/mL). Achieving a CI of <0.2 while at a four-fold lower dose is a significant improvement.

### Synergistic Combination of Turmeric (95 wt % Curcuminoids) Plus Ginger or Carrageenan

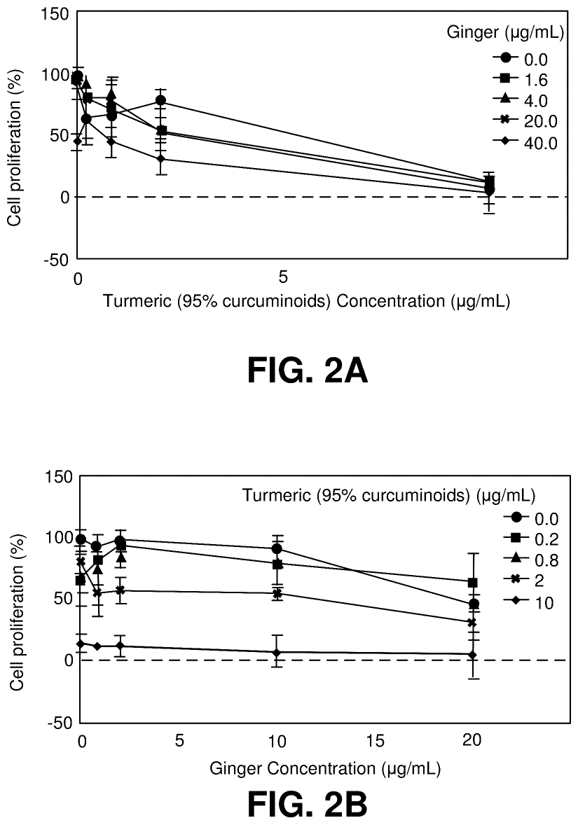

In preliminary experiments the IC₅₀ values for turmeric (95 wt % curcuminoids) and ginger alone were approximately 5.6 μg/ml and 19 μg/ml, respectively, on HeLa cervical cancer cells. Turmeric enhances the growth inhibitory effect of ginger on HeLa cells. The CI for the combination of turmeric (95 wt % curcuminoids) 2 μg/ml and ginger 10 μg/ml was approximately 0.53, indicating strong synergy. See FIG. 2A and FIG. 2B.

### Synergistic Combination of Turmeric (95 wt % Curcuminoids) Plus Carrageenan

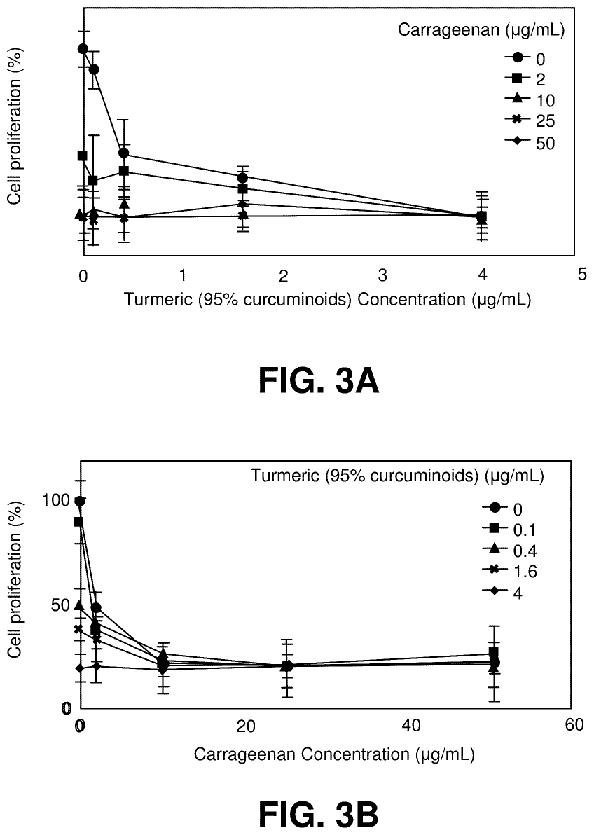

Since carrageenan interferes with HPV16 attachment to the cell and later steps of viral replication, the growth inhibitory effects of combinations of carrageenan and turmeric (95 wt % curcuminoids) on HeLa cells was examined. In preliminary experiments, the IC₅₀ values for turmeric (95 wt % curcuminoids) and carrageenan alone (after sonication) were approximately 0.4 μg/ml and 2.0 μM, respectively, on HeLa cervical cancer cells. Turmeric enhances the growth inhibitory effect of carrageenan on the human cervical cancer cell line HeLa. The CI for the combination of turmeric (95 wt % curcuminoids) 0.1 μg/ml and carrageenan 2 μg/ml was approximately 0.78 indicating moderate synergy. Cell viability data is shown in FIG. 3A and FIG. 3B.

### Synergistic Combination of Kava and Ginger

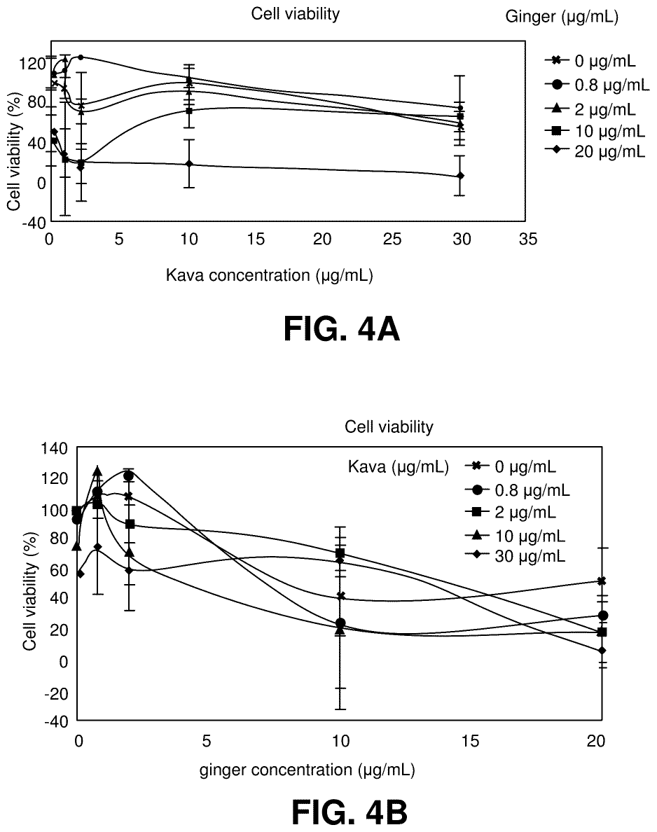

In preliminary experiments, the IC₅₀ values for kava and ginger alone were approximately 7 μg/ml and 34 μM, respectively, on H29 colon cancer cells. Cell viability data is shown in FIG. 4A and FIG. 4B. When increasing concentrations of ginger were combined with increasing concentrations of kava (FIG. 4A, FIG. 4B), the percent viable cells decreased substantially beyond the effect of either agent alone. At a dose of 2 μg/mL kava, viable cells decreased from 76.90% (with kava alone) to 124.21%, 71.20%, 21.64% and 19.19% when combined with ginger 0.8, 2, 10 and 20 μg/mL, respectively (p<0.01). Cell viability was measured after 96 hours.

The combination index (CI) for ginger 10 μg/mL plus kava 0.8 μg/mL was 0.40, indicating strong synergy, while ginger 10 μg/mL and kava 2 μg/mL produced a CI of 0.79, indicating moderate synergy. The marked reduction in viable cells demonstrates that ginger enhances the growth-inhibitory effect of kava in colon cancer cells.

### Synergistic Combination of Dihydromethysticin (DHM) and Turmeric (95 Wt % Curcuminoids)

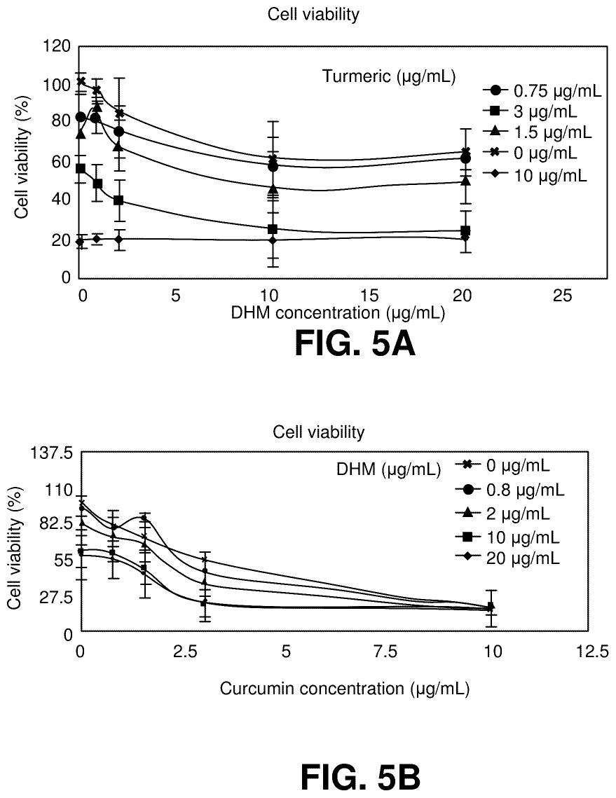

The IC₅₀ values for DHM and turmeric alone against HT29 colon cancer cells were approximately 30 μg/mL and 3.9 μg/mL, respectively. When increasing concentrations of turmeric were combined with increasing concentrations of DHM (FIG. 5A, FIG. 5B), the percent viable cells decreased substantially more than with either agent alone. At a dose of 2 μg/mL DHM, the percent viable cells decreased from 84.92% with DHM alone to 74.71% with curcumin 0.75 μg/mL, 67.63% with curcumin 1.5 g/mL, 39.34% with curcumin 3 μg/mL, and 19.16% with curcumin 10 μg/mL (p<0.01). Cell viability was measured after 96 hours.

Combination index values indicated moderate to strong synergy. Turmeric (1.5 μg/mL) plus DHM (2 μg/mL) yielded CI=0.788 (moderate synergy). Turmeric (3 μg/mL) plus DHM (2 μg/mL) yielded CI=0.563 (strong synergy). Turmeric (3 μg/mL) plus DHM (0.8 μg/mL) produced CI=0.769 (moderate synergy). These results show that turmeric strongly enhances the inhibitory activity of DHM in colon cancer cells, reducing viable cell numbers far more than either compound alone.

### Synergistic Combinations of DHM and 5-FU

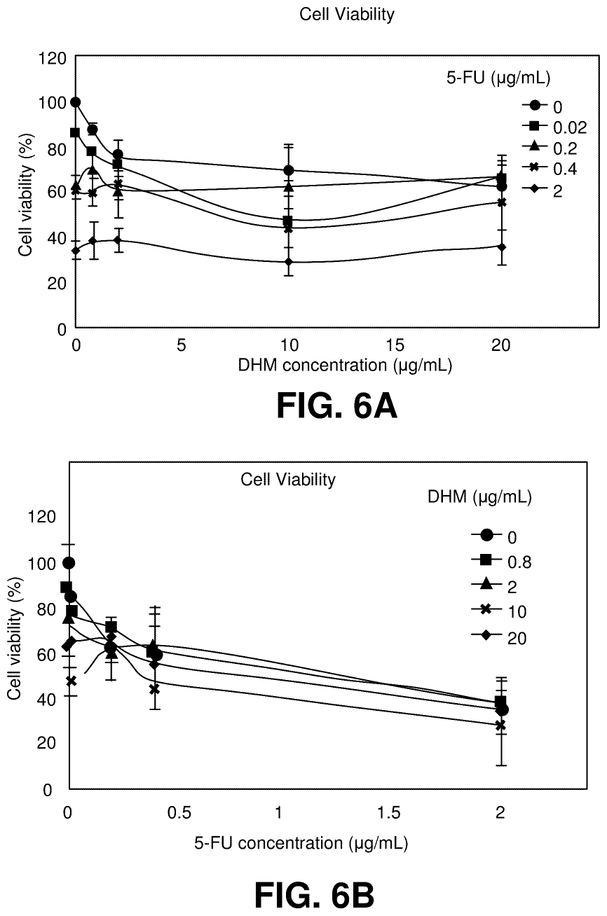

The IC₅₀ values for DHM and 5-fluorouracil (5-FU) alone against HT29 colon cancer cells were approximately 35 μg/mL and 1 μg/mL, respectively. When increasing concentrations of 5-FU were combined with increasing concentrations of DHM (FIG. 6A, FIG. 6B), the percent viable cells decreased substantially more than with either agent alone. Cell viability was measured after 96-hour treatment. At a dose of 10 μg/mL DHM, the percent viable cells decreased from 69.60% with DHM alone to 47.32% with 5-FU 0.02 μg/mL, 62.62% with 5-FU 0.2 μg/mL, 43.93% with 5-FU 0.4 μg/mL, and 29.04% with 5-FU 2 μg/mL (p<0.01). Cell viability was measured after 96 hours. Data points represent mean values from replicate experiments.

The combination index (CI) for DHM 10 μg/mL combined with 5-FU 0.02 μg/mL was less than 0.3 (approximately 0.20-0.30), indicating very strong synergy between DHM and 5-FU in inhibiting colon cancer cell growth. Synergistic effects were also observed at lower DHM concentrations: DHM 2 g/mL combined with 5-FU 0.2 μg/mL produced a CI of 0.56 (strong synergy) (p<0.01). At the lowest DHM concentration tested, DHM 0.8 μg/mL combined with 5-FU 0.4 μg/mL yielded a CI of 0.87 (moderate synergy). These results demonstrate that DHM exhibits synergistic activity with 5-FU across a range of concentrations.

### Methods of Treatment

The disclosed combinations may be used as therapeutic to treat cancerous cells, including HPV-related cancerous cells. In one embodiment, cervical cancer is treated. In another embodiment, colon cancer is treated. The combinations may be administered orally (e.g. as an oral supplement) or topically (e.g. a liquid or cream administered to the cervix or colon).

The disclosed combinations may be used as a preventative to treat precancerous cells, including HPV-related cancerous cells. These include cancers caused by human papillomavirus (HPV), cytomegalovirus (CMV), herpes simplex virus (HSV), Epstein-Barr virus (EBV), hepatitis B virus (HBV), hepatitis C virus (HCV), or human immunodeficiency virus (HIV). In one embodiment, the disclosed methods or compositions are used on a subject that has been diagnosed with at least one of the aforementioned conditions. In one embodiment, cervical precancerous are treated. In another embodiment, colon precancerous cells are treated. The combinations may be administered orally (e.g. as an oral supplement) or topically (e.g. a liquid or cream administered to the cervix or colon).

In any of the aforementioned treatment methods, the composition may be formulated as a solid, cream, gel, anal suppository or vaginal suppository. The composition may be incorporated into a vaginal device (e.g. a tampon, a ring, a pessary, a sponge, a foam, a male condom or a female condom etc.) or a device that is inserted into an oral cavity or an anal cavity, or placed in contact with the skin (e.g. an adhesive patch). The composition may also be formulated as a tablet, a foam, a spray, a film, a sponge, an emulsion, a solution, a nanofiber, a suspension, a particle, a nanoparticle, a bio-adhesive, or an oral rinse. The two compounds in the composition may be delivered simultaneously (e.g. as a single composition) or sequentially (e.g. as two separate compositions delivered in a relatively short period of time) provided both compounds are simultaneously present in a patient's body.

The composition may be co-formulated with one or more of a lipophilic carrier, a hydrophilic carrier, a muco-adhesive agent, a solubilizing agent, a stabilizer, an emulsifier, a buffer, a lubricant and/or a filler. A lipophilic carrier is a substance that dissolves in fats, oils and lipids. Examples include mineral oil, olive oil, caprylic/capric triglyceride, medium-chain triglycerides (MCT), castor oil, mineral oil, lechithin, vegetable oils, corn oil, sesame oil, ethyl oleate, oleic acid. A hydrophilic carrier is a substance that dissolves in water. Examples include water, glycerin, propylene glycol, ethanol, hydroxypropyl methylcellulose (HPMC) and polyethylene glycol (PEG). A muco-adhesive agent is a composition that helps adhere to mucosal surfaces. Examples include carbopol, chitosan, hyaluronic acid, sodium alginate, hydroxypropyl methylcellulose (HPMC), poly(acrylic acid), sodium carboxymethylcellulose, hydroxypropyl cellulose. A solubilizing agent is a substance that aids in dissolution. Examples include polysorbates (e.g. polysorbate 80), cyclodextrins, polyethylene glycol (PEG), PEG-40 hydrogenated castor oil, propylene glycol, ethanol, cremophor EL, sodium lauryl and sulfate. A stabilizer helps maintain stability of the composition. Examples include lechithin, sodium stearoyl lactylate, polysorbates (Tween 20, Tween 80, polysorbate 60), lecithin, sodium citrate, tris buffer, sodium metabisulfite, ascorbic acid, Ethylenediaminetetraacetic acid (EDTA), citric acid, butylated hydroxytoluene (BHT), tocopherols. Examples of emulsifiers include polysorbate 80, lecithin, sodium lauryl sulfate, cetyl alcohol, sorbitan monooleate and polyethylene glycol stearate. A buffer is a compound that maintains the pH of the composition. Examples include phosphate buffer (e.g. sodium phosphate), citrate buffer (e.g. citric acid), carbonate buffer (e.g. sodium bicarbonate), acetate buffer, tris buffer, HEPES buffer. A lubricant is a compound that reduces friction. Examples include silicone oil, glycerin, mineral oil, magnesium stearate, talc, stearic acid, stearic acid, calcium stearate, polyethylene glycol and colloidal silicon dioxide. A filler is a compound that adds bulk to the composition. Examples include microcrystalline cellulose, lactose, starch, calcium phosphate, maltodextrin, sorbitol and dicalcium phosphate.

### Growth Inhibitory Effect

The growth inhibitory effects of the most active phytochemicals on cervical keratinocytes derived from a precancerous lesion (W12; CIN grade 1) were determined. W12E cells contain full-length HPV16 episomes and W12I contain integrated HPV16 DNA. W12I clones can further be categorized into type 1 integration (3-5 genomes) and type 2 integration (30-60 genomes). W12 subclone 20850 contains approximately 5-100 extrachromosomal HPV16 genomes. After culture in vitro, the W12 cells generated the clones 20822 and 201402 containing three and five HPV16 genomes, respectively, that are recombined with the cellular DNA (type 1 integrated clones). In addition, the W12 cells generated clones 20861 and 20862, which contain many HPV16 genomes, 30 or 60 recombined viral genomes (viral genomes plus junction fragments), respectively, (type 2 integration). The W12 type 1 and 2 clones resemble the HPV DNA integration state of cancer derived cell lines, SiHa (1-2 HPV16 copies; CIN grade 2) and CaSki (500 HPV16 copies; CIN grade 3), respectively. Cells were exposed to increasing concentration of agents for 120 h and the number of viable cells determined by the MTT assay. The results are depicted in Table 2.

TABLE 2
Growth inhibition IC₅₀ values on W12 clones

| Compound/ | W12 20850 | W12 20822 | W12 20862 | W12 20861 |
|---|---|---|---|---|
| Extract | (Episomal) | (Type 1) | (Type 2) | (Type 2) |
| Tanshinone | 2.3 μg/mL | 2.0 μg/mL | 3.1 μg/mL | 1.4 μg/mL |
| IIA | (7.8 μM) | (6.8 μM) | (10.6 μM) | (4.8 μM) |
| DHM | 24.7 μg/mL | 10.2 μg/mL | 12.8 μg/mL | 12.4 μg/mL |
| | (89.4 μM) | (36.9 μM) | (46.3 μM) | (44.9 μM) |
| Turmeric | 4.7 μg/mL | 2.8 μg/mL | 2.0 μg/mL | 1.6 μg/mL |
| (95%) | | | | |
| Ginger | 13.7 μg/mL | 11.6 μg/mL | 21.6 μg/mL | 15.5 μg/mL |

These data indicate the compounds are effective against precancerous cervical cells. Accordingly, the disclosed synergistic combinations that contain one of these compounds is also effective against precancerous cells.

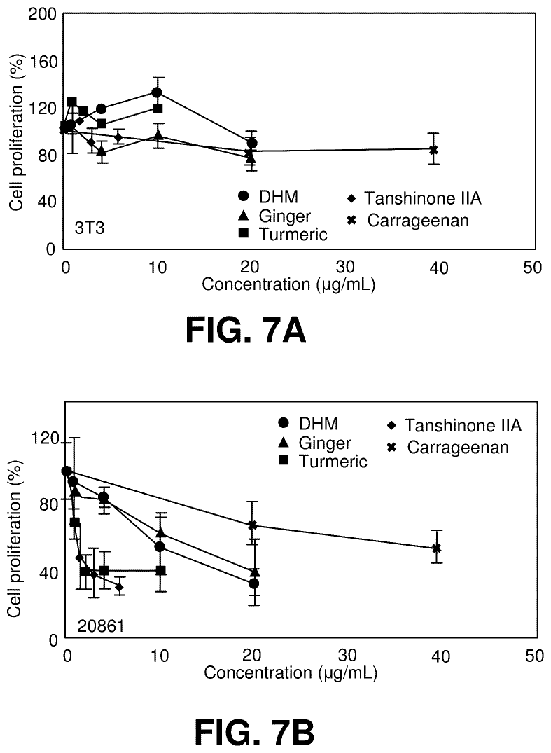

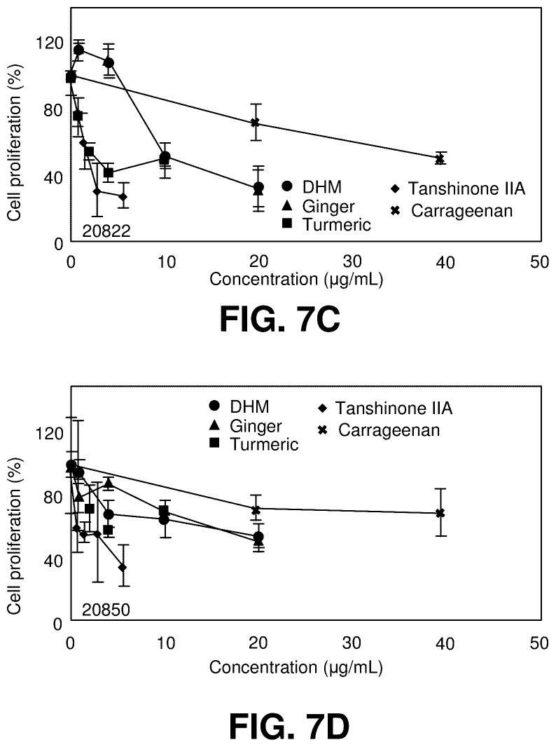

Referring to FIG. 7A, FIG. 7B, FIG. 7C and FIG. 7D, the relative order of activity of the phytochemicals on W12 cells is similar to that on HeLa cells. For type 2 integrant (20861) cells, the descending order of activity is: (IC₅₀ values in parentheses): (compounds) tanshinone IIA (1.4 μg/mL; 4.8 μM); DHM (12.4 μg/mL; 44.9 μM); (fractions) turmeric; (1.6 μg/mL); and ginger (15.5 μg/mL).

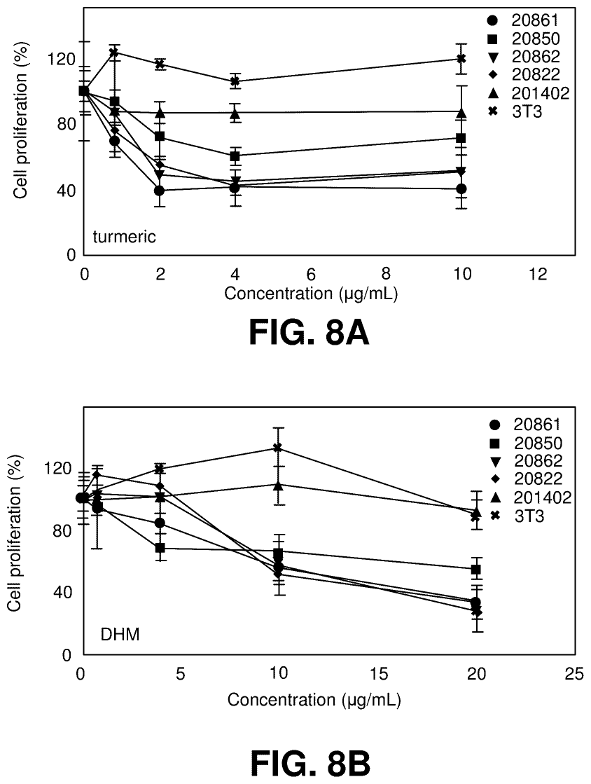

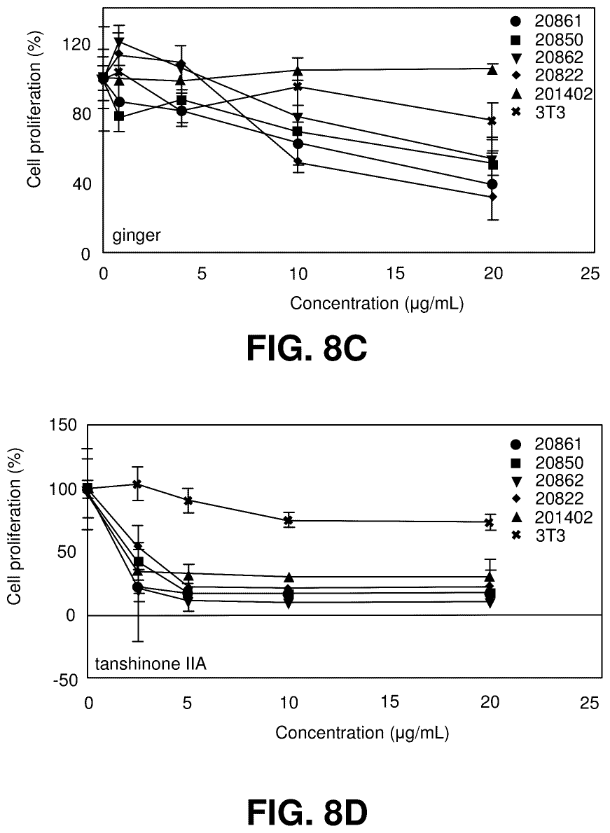

The three W12 cell lines displayed similar patterns of activity. However, there were notable differences. Ginger was highly active on 20822 cells; and carrageenan (after sonication), on 20850 cells; in a repeat experiment, carrageenan exhibited the inverse pattern of activity. The activity on type 2 20862 cells was also assayed. The order of activity was similar to that on type 2 20861 cells. Turmeric was the most active fraction (1.96 μg/mL). Turmeric and tanshinone IIA showed significant activity on 20850 (episomal) cells at low concentrations: turmeric 10 μg/mL (p=0.0402) and tanshinone IIA 2.5 μM (p=0.0058). Referring to FIG. 8A, FIG. 8B, FIG. 8C and FIG. 8D, the relative sensitivity of the W12 cell lines to some of the most active phytochemicals, including turmeric, DHM, ginger, and tanshinone IIA, is illustrated. Tanshinone IIA exhibited potent activity against the growth of W12 cells.

Effects of tanshinone IIA on expression of p53 and HPV16 mRNAs

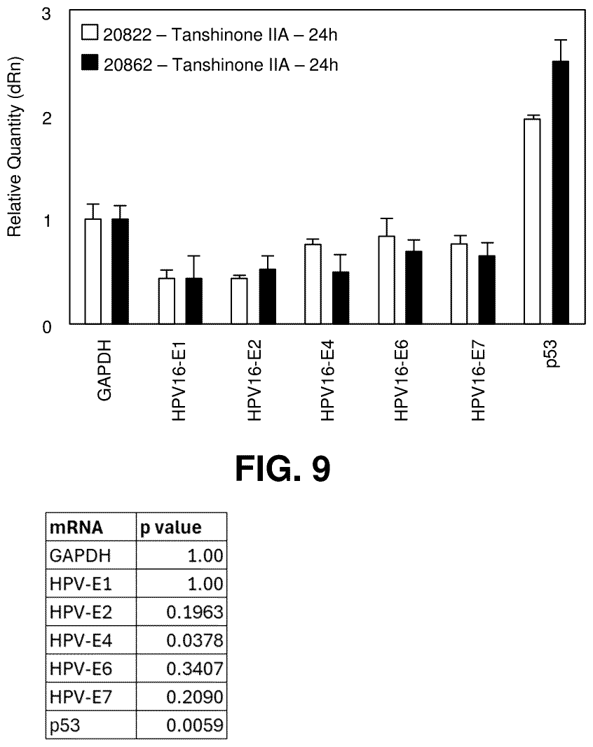

To investigate the mode of action of tanshinone IIA, real-time polymerase chain reaction (RT PCR) experiments were performed for p53 and HPV16 mRNAs on W12 type 1 (20822) and type 2 (20862) integrant cells. The results are displayed in FIG. 9.

The sensitive tool RT-PCR showed that tanshinone IIA activated the expression of p53 at 24 h in the two W12 cell types tested, type 1 and 2 integrants (relative fold: 20822: 1.96; 20862:2.53). The difference between the relative levels of p53 mRNAs in the 2 cell types (20822 vs. 20862) at 24 h is significant (p<0.01) (Table 3). W12 cells were treated with tanshinone IIA at 5 μM for 24 hours; extracts were prepared and analyzed by RT-PCR. Fold change indicates relative expression in tanshinone IIA treated versus control cells. The activation was greater on type 2 compared to type 1 integrant cells, which may relate to the state of integration of the HPV16 genome.

TABLE 3
Effect of tanshinone IIA on the relative level
of HPV16 and p53 mRNAs in W12 cells.

| W12 cells type | type 1 | type 2 |
|---|---|---|
| W12 cells clone number | 20822 | 20862 |
| Relative level of mRNA | 1 +/− 0.14 | 1 +/− 0.13 |
| Gapdh | | |
| E1 | 0.46 +/− 0.05 | 0.46 +/− 0.19 |
| E2 | 0.44 +/− 0.02 | 0.54 +/− 0.11 |
| E4 | 0.78 +/− 0.03 | 0.51 +/− 0.15 |
| E6 | 0.84 +/− 0.17 | 0.72 +/− 0.09 |
| E7 | 0.78 +/− 0.07 | 0.66 +/− 0.12 |
| p53* | 1.96 +/− 0.04 | 2.53 +/− 0.18 |

*separate experiment

To explore an effect on p53 protein levels, the effect of tanshinone IIA on the mRNA levels of genes responsive to p53 was monitored, such as p21 (Cyclin Dependent Kinase Inhibitor 1A) and MDM2 (MDM2 Proto-Oncogene). MDM2, which encodes a nuclear-localized E3 ubiquitin ligase, is the major negative regulator of p53. In preliminary experiments on W12 20822 (type 1 integrant) cells tanshinone IIA activated the expression of p21 (1.41-fold) and repressed the expression of MDM2 (0.78-fold), at 24 h. The effect on MDM2 is the inverse of the effect on p53.

Effect of Tanshinone IIA on the Expression of HPV16 mRNAs

Concerning HPV16 mRNAs, tanshinone IIA repressed the expression of HPV16 mRNAs (E1, E2, E4, E6, E7) in both W12 cell types tested (type 1 and 2 integrants). The relative fold reduction is shown in FIG. 9 and Table 3. The early genes were more sensitive than the late gene E4. The reduction in expression of the early gene E2 was greater in 20822 (0.44) vs. 20862 (0.54) cells; while the reduction in expression of the late genes (E4, E6, E7) was greater in 20862 versus 20822 cells. The difference between the relative levels of HPV16 mRNAs in the 2 cell types, 20822 vs. 20862, at 24 h, is significant for E4 (p<0.01) (Table 3).

The two cell types (types 1 and 2 integrants) were about equally sensitive by RT-PCR assay. This corresponds to the relative growth inhibitory effect (IC₅₀ values in parentheses): 20822 (2.1 μM); 20862 (2.8 μM).

### Bioelectric Signaling

Because the Na⁺/K⁺-ATPase plays a role in the development and progression of cervical cancer, the effect of turmeric on the activity of the enzyme was assayed. Turmeric inhibited the activity of the enzyme with the IC₅₀ of curcumin being 2 μM.

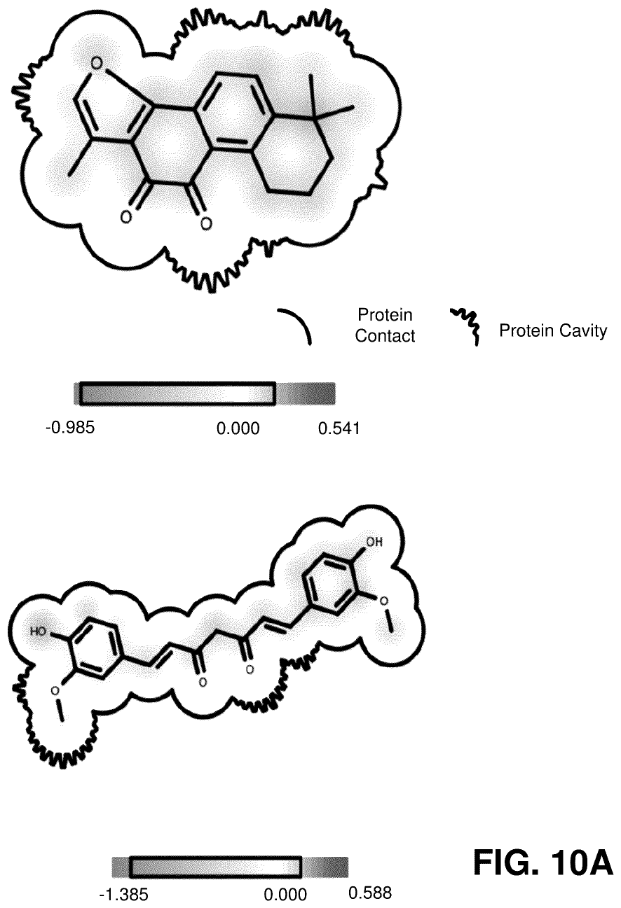

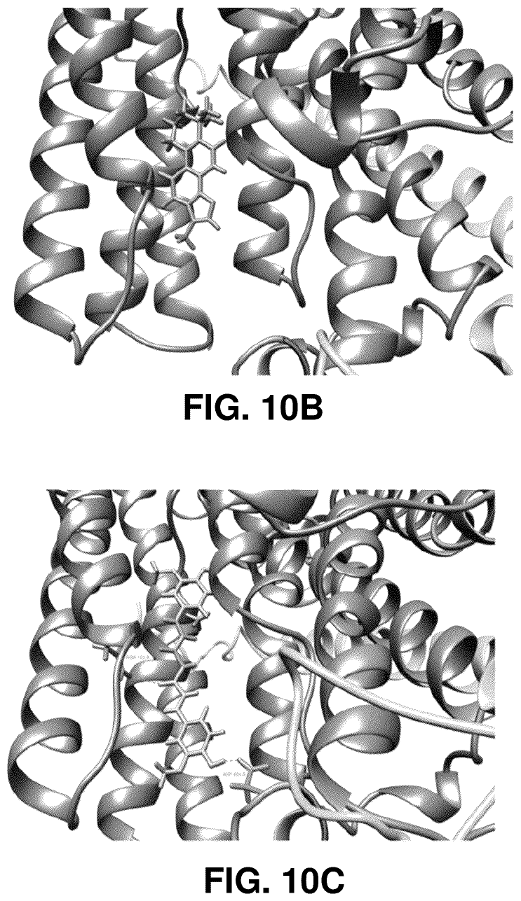

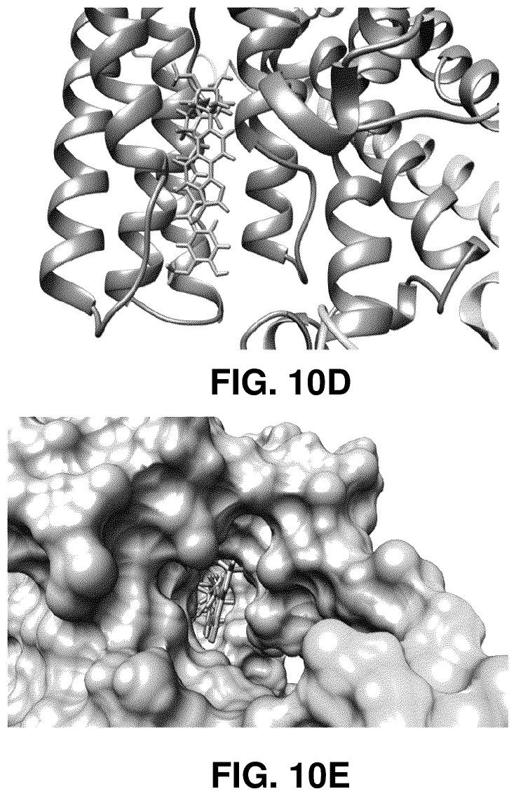

To gain insight into the mode of action of tanshinone IIA and curcumin, alone and in combination, we assessed their binding to the Na⁺/K⁺-ATPase, using molecular docking. For tanshinone IIA (FIG. 10B), the docking score was −10.45. The majority of the score comes from the shape evaluation (FIG. 10A), which means the ligand shape is fit to the protein active site (docking pocket). The hydrogen bond score and the residue fingerprint show no H-bonds formed between the protein and tanshinone IIA. Curcumin (FIG. 10C) binds with high affinity; the docking score was −12.74. Curcumin forms hydrogen bonds with the residues ASN 122 and ASP 884 of protein chain A.

To better understand the synergistic effects of tanshinone IIA and curcumin, the binding of the combination to the Na⁺/K⁺-ATPase was assayed. The best poses of the two ligands aligned in the Na⁺/K⁺-ATPase active site and protein are shown as the secondary structure in FIG. 10D; the two ligands aligned in the protein pocket with different patterns. The protein is present with the solvent-accessible surface, which is generated by Chimera in default parameters. This figure shows that tanshinone IIA fits in the deep place of the protein pocket. The tanshinone IIA docked position is well aligned with that of curcumin. This shows that curcumin and tanshinone IIA possibly interact with the protein at similar positions. However, tanshinone IA did not share similar protein-ligand interactions (H-bonds) with curcumin to the Na⁺/K⁺-ATPase. FIG. 10E displays the binding of tanshinone IIA and curcumin to the Na⁺/K⁺-ATPase with solvation.

### Materials and Methods

Turmeric powder (95 wt %) (Curcuma longa L.) was purchased from Naturex (Avignon, France), Item #: 140500; the extract contained approximately 74 wt % curcumin (diferuloylmethane): typical range: curcumin (72-75 wt %); bisdemethoxycurcumin (3-4 wt %); demethoxycurcumin (16-17 wt %);

Turmeric powder (90 wt %); 71.86 wt % curcumin; 89.88 wt % total curcuminoids; 96.88 wt % total curcuminoid complex (consisting of curcumin, desmethoxycurcumin, bis-demethoxycurcumin and volatile oils of turmeric rhizome) (Dolcas Biotech LLC) was purchased from PCCA (Houston, TX). Supplier labeled this composition as 95% curcuminoids but analysis indicated only about 90% curcuminoids.

Ginger (Zingiber officinale Roscoe) root soft extract (20.5 wt % total pungent compounds calculated as gingerols and shogaol, Item #: 143010 from Naturex (Avignon, France); Extraction was with ethyl acetate and contained≤5 wt % moisture and less than 10 mg/kg residual ethyl acetate. Total gingerols+shogaols ≥20 wt % (HPLC), 6-gingerol (˜8-10 wt %), 8-gingerol (˜2-3 wt %), 10-gingerol (3-4 wt %), shogoals (˜2-3 wt %), remainder being ginger polysaccharides, proteins, and native non-volatile constitutes.

Kava (Piper methysticum G. Forst; 31.2 wt % kavalactones, Item #: 332679 from Naturex (Avignon, France) sometimes referred to as Nat-k. In one embodiment, the kava is dihydrokavain (40-45 wt %), kavain (15-20 wt %), dihydromethysticin (15-20 wt %), methysticin (>0 but ≤5 wt %), yangonin (5-10 wt %), and demethoxyyangonin (3-5 wt %) with the remainder (about 70 wt %) being native kava constituents.

The following compounds were purchased from Sigma (St. Louis, MO): tanshinone IIA (>/=97 wt %); CAS number 568-72-9.

Lambda carrageenan (red seaweed: Chondrus crispus Stackh) plant mucopolysaccharide; alpha-kavain (>/=95 wt %).

Dihydroxymethysticin (DHM) (>/=85 wt %) was purchased from Chromadex (CA). DHM is a kavalactone from kava.

Cell culture: cancer cells: HeLa human cervical cancer cells were obtained from ATCC. HT29 (p53 positive; similar to enterocytes from the small intestine) colon cancer cells were obtained from ATCC (Manassas, VA). HeLa cells were grown in Dulbecco's Modified Eagle's medium (DMEM) (Gibco BRL Life Technologies, Inc., Rockville, MD) containing 10% (v/v) fetal bovine serum (FBS) (Gibco BRL), plus Pen Strep (Gibco), while HT29 were maintained in McCoy's media plus 10% FBS, plus Pen Strep (Gibco); at 37° C., 5% CO₂.

Proliferation Assay: Cells were seeded in 96 well plates at 1000 cells per well. After incubating for 24 h, phytochemicals were added. For the W12 assays, plant solutions were sonicated for 10 min each to increase their solubility. This may degrade the structure of carrageenan. For the combination experiments, plant solutions were sonicated for 10 min, two times, before addition to cell cultures. The sensitivity of the various cell lines to herbal agents was assayed using the MTT assay or the EZQUANT assay. Values were normalized to those of cells treated with vehicle controls. For the EZQUANT assay, absorbance was read at 420 nM (for the MTT assay, at 600 nM) and corrected for the background values. IC₅₀ values were estimated from the graphs, using a line for 50% cell proliferation. In the second and later sets of experiments on W12 cells, treatments were prepared from a lower concentration of stock solutions; the resulting IC₅₀ values were lower.

Calculating the Combination Index: To assess possible synergistic effects, cells were treated with all combinations of four concentrations of each of the agents tested and a solvent control, as previously described in Planta Med. 72, 1200-1206, 2006.

The median effect principle was used to analyze the combination assays. Variable ratios of drugs were used and assumed mutually exclusive equations to determine the Combination Index (CI). IC₅₀ values determined from the graphs were used to obtain combination index values; for agents 1 and 2: CI=[IC₅₀ (agent 1+agent 2)/(IC₅₀ (agent 1 alone)]+ [IC₅₀ (agent 2+agent 1)/IC₅₀ (agent 2 alone)]. CI and its corresponding effects are as follows: >1.3, antagonism; 1.1-1.3, moderate antagonism; 0.9-1.1, additive effect; 0.8-0.9, slight synergism; 0.6-0.8, moderate synergism; <0.6, synergism.

W12 subclones: W12E (episomal, 20850) and W12I (integrant: type 1:20822, 201402; type 2:20861, 20862) and J2 3T3 mouse embryo fibroblasts (feeder cells). The W12 cells were derived from a cervical intraepithelial lesion (CIN grade 1); the subclone 20850 contains approximately 5-100 extrachromosomal HPV16 genomes. After culture in vitro, the W12 cells generated the clones 20822 and 201402 containing three and five HPV16 genomes, respectively, that are recombined with the cellular DNA (type 1 integrated clones). In addition, the W12 cells generated clones 20861 and 20862, which contain many HPV16 genomes, 30 or 60 recombined viral genomes (viral genomes plus junction fragments), respectively, (type 2 integration).

### Definitions

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Consisting essentially of: As used here, the phrase “consisting essentially of” refers to a combination of components that contains the specified components plus additives provided additives do not materially affect the basic and novel characteristics of the combination of components. The additives are deemed to not have a material effect if the combination index (CI), with and without the additives, remains within 10% of one another when the combination is tested on HeLa cells (for cervical cancer) or on HT29 cells (for colon cancer).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.
A1. A method of treating cervical cancer or precancer cells in a subject in need thereof, the method comprising:
   administering to the subject a therapeutically effective amount of a combination of turmeric and tanshinone IIA, wherein the combination provides a synergistic combination index (CI) of 0.8 or less on HeLa cells.
A2. The method as recited in claim A1, wherein the combination is free of epigallocatechin-3-gallate (EGCG).
A3. The method as recited in claim A1, wherein the combination is free of resveratrol.
A4. The method as recited in claim A1, wherein the combination is free of both epigallocatechin-3-gallate (EGCG) and resveratrol.
A5. The method as recited in any one of claims A1-A4, wherein the combination provides a synergistic combination index (CI) of 0.7 or less.
A6. The method as recited in any one of claims A1-A4, wherein the combination provides a synergistic combination index (CI) of 0.6 or less.
A7. The method as recited in any one of claims A1-A4, wherein the combination provides a synergistic combination index (CI) of 0.5 or less.
A8. The method as recited in any one of claims A1-A4, wherein the combination provides a synergistic combination index (CI) of 0.4 or less.
A9. The method as recited in any one of claims A1-A4, wherein the combination provides a synergistic combination index (CI) of 0.3 or less.
A10. The method as recited in any one of claims A1-A4, wherein the combination provides a synergistic combination index (CI) of 0.2 or less.
A11. The method as recited in any one of claims A1-A4, wherein the combination provides a synergistic combination index (CI) of 0.1 or less.
A12. The method as recited in any one of claims A1-A11, wherein the combination consists essentially of the tanshinone IIA and the turmeric.
A10. The method as recited in any one of claims A1-A12, wherein the synergistic combination index (CI) is measured when the HeLa cells are exposed to a tanshinone IIA concentration of >0 μM to ≤10 UM and a turmeric concentration of >0 μg/mL to ≤8 μg/mL.
A14. The method as recited in any one of claims A1-A12, wherein the synergistic combination index (CI) is measured when the HeLa cells are exposed to a tanshinone IIA concentration of >0 μM to ≤5 μM and a turmeric concentration of >0 μg/mL to ≤4 μg/mL.
A15. The method as recited in any one of claims A1-A12, wherein the synergistic combination index (CI) is measured when the HeLa cells are exposed to a tanshinone IIA concentration of >0 μM to ≤1 μM and a turmeric concentration of >0 μg/mL to ≤1 μg/mL.
A16. The method as recited in any one of claims A1-A15, wherein the turmeric is at least 90 wt % curcuminoids selected from the group consisting of curcumin, desmethoxycurcumin, bis-demethoxycurcumin and combinations thereof.
A17. The method as recited in any one of claims A1-A15, wherein the turmeric is at least 95 wt % curcuminoids selected from the group consisting of curcumin, desmethoxycurcumin, bis-demethoxycurcumin and combinations thereof.
A18. The method as recited in any one of claims A1-A17, wherein the combination is administered orally.
A19 The method as recited in any one of claims A1-A17, wherein the combination is administered orally and the combination is formulated as a tablet or a liquid.
A20. The method as recited in any one of claims A1-A17, wherein the combination is administered topically to a portion of the subject.
A21. The method as recited in any one of claims A1-A17, wherein the combination is administered topically and the combination is formulated as a liquid, foam or a cream.
A22. The method as recited in any one of claims A1-A17, wherein the combination is administered topically and the combination is formulated as a vaginal suppository.
A23. The method as recited in any one of claims A1-A17, wherein the combination is incorporated into a vaginal device selected from the group consisting of a tampon, a pessary, a sponge, a male condom and a female condom.
A24. The method as recited in any one of claims A20-A23, wherein the therapeutically effective amount is selected to provide a tanshinone IIA concentration of >0 M to ≤5 M and a turmeric concentration of >0 μg/mL to ≤4 μg/mL to the portion of the subject.
A25. The method as recited in any one of claim A1-A24, wherein the turmeric and the tanshinone IIA are administered simultaneously.
A26. The method as recited in any one of claim A1-A24, wherein the turmeric and the tanshinone IIA are administered sequentially such that the turmeric and the tanshinone IIA are simultaneously present in the subject.
B1. A composition of matter comprising turmeric and tanshinone IIA, wherein the composition of matter is free of both epigallocatechin-3-gallate (EGCG) and resveratrol.
B2. The composition of matter as recited in claim B1, wherein the turmeric is at least 90 wt % curcuminoids selected from the group consisting of curcumin, desmethoxycurcumin, bis-demethoxycurcumin and combinations thereof.
B3. The composition of matter as recited in claim B1, wherein the turmeric is at least 95 wt % curcuminoids selected from the group consisting of curcumin, desmethoxycurcumin, bis-demethoxycurcumin and combinations thereof.
B4. The composition of matter as recited in any one of claims B1-B3, wherein the composition consists essentially of the tanshinone IIA and the turmeric.
B5. The composition of matter as recited in any one of claims B1-B4, wherein the composition provides a synergistic combination index (CI) of 0.8 or less on HeLa cells.
B6. The composition of matter as recited in any one of claims B1-B4, wherein the composition provides a synergistic combination index (CI) of 0.7 or less on HeLa cells.
B7. The composition of matter as recited in any one of claims B1-B4, wherein the composition provides a synergistic combination index (CI) of 0.6 or less on HeLa cells.
B8. The composition of matter as recited in any one of claims B1-B4, wherein the composition provides a synergistic combination index (CI) of 0.5 or less on HeLa cells.
B9. The composition of matter as recited in any one of claims B1-B4, wherein the composition provides a synergistic combination index (CI) of 0.4 or less on HeLa cells.
B10. The composition of matter as recited in any one of claims B1-B4, wherein the composition provides a synergistic combination index (CI) of 0.3 or less on HeLa cells.
B11. The composition of matter as recited in any one of claims B1-B4, wherein the composition provides a synergistic combination index (CI) of 0.2 or less on HeLa cells.
B12. The composition of matter as recited in any one of claims B1-B4, wherein the composition provides a synergistic combination index (CI) of 0.1 or less on HeLa cells.
B13. The composition of matter as recited in any one of claims B1-B12, wherein the composition of matter is formulated as a liquid, foam or a cream.
B14. The composition of matter as recited in any one of claims B1-B12, wherein the composition is incorporated into a vaginal device selected from the group consisting of a tampon, a pessary, a sponge, a male condom and a female condom.
B15. The composition of matter as recited in any one of claims B1-B12, wherein the composition is formulated as a vaginal suppository.
B16. The composition of matter as recited in any one of claims B1-B12, wherein the composition of matter is formulated as tablet.
C1. A method of treating cervical cancer or precancer cells in a subject in need thereof, the method comprising:
   administering to the subject a therapeutically effective amount of a combination of ginger and turmeric, wherein the combination provides a synergistic combination index (CI) of 0.8 or less on HeLa cells.
C2. The method as recited claim C1, wherein the combination provides a synergistic combination index (CI) of 0.7 or less.
C3. The method as recited claim C1, wherein the combination provides a synergistic combination index (CI) of 0.6 or less.
C4. The method as recited claim C1, wherein the combination provides a synergistic combination index (CI) of 0.5 or less.
C5. The method as recited claim C1, wherein the combination provides a synergistic combination index (CI) of 0.4 or less.
C6. The method as recited claim C1, wherein the combination provides a synergistic combination index (CI) of 0.3 or less.
C7. The method as recited claim C1, wherein the combination provides a synergistic combination index (CI) of 0.2 or less.
C8. The method as recited claim C1, wherein the combination provides a synergistic combination index (CI) of 0.1 or less.
C9. The method as recited in any one of claims C1-C8, wherein the combination consists essentially of the ginger and the turmeric.
C10. The method as recited in any one of claims C1-C9, wherein the synergistic combination index (CI) is measured when the HeLa cells are exposed to a ginger concentration of >0 μg/mL to ≤10 μg/mL and a turmeric concentration of >0 Mg/mL to ≤5 Mg/mL.
C11. The method as recited in any one of claims C1-C9, wherein the synergistic combination index (CI) is measured when the HeLa cells are exposed to a ginger concentration of >0 g/mL to ≤10 μg/mL and a turmeric concentration of >0 μg/mL to ≤2 μg/mL.
C12. The method as recited in any one of claims C1-C11, wherein the turmeric is at least 90 wt % curcuminoids selected from the group consisting of curcumin, desmethoxycurcumin, bis-demethoxycurcumin and combinations thereof.
C13. The method as recited in any one of claims C1-C11, wherein the turmeric is at least 95 wt % curcuminoids selected from the group consisting of curcumin, desmethoxycurcumin, bis-demethoxycurcumin and combinations thereof.
C14. The method as recited in any one of claims C1-C13, wherein the ginger is ≥20 wt % gingerols and shogaols, 8-10 wt % 6-gingerol, 2-3 wt % 8-gingerol, 3-4 wt % 10-gingerol, 2-3 wt % shogoals with the remainder being ginger polysaccharides, proteins, and native non-volatile components.
C15. The method as recited in any one of claims C1-C14, wherein the combination is administered orally.
C16 The method as recited in any one of claims C1-C14, wherein the combination is administered orally and the composition is formulated as a tablet or a liquid.
C17. The method as recited in any one of claims C1-C14, wherein the combination is administered topically to a portion of the subject.
C18. The method as recited in any one of claims C1-C14, wherein the combination is administered topically and the combination is formulated as a liquid, foam or a cream.
C19. The method as recited in any one of claims C1-C14, wherein the combination is administered topically and the combination is formulated as a vaginal suppository.
C20. The method as recited in any one of claims C1-C14, wherein the combination is incorporated into a vaginal device selected from the group consisting of a tampon, a pessary, a sponge, a male condom and a female condom.
C21. The method as recited in any one of claims C17-C20, wherein the therapeutically effective amount is selected to provide a ginger concentration of >0 μg/mL to ≤10 μg/mL and a turmeric concentration of >0 μg/mL to ≤4 μg/mL to the portion of the subject.
C22. The method as recited in any one of claim C1-C21, wherein the turmeric and the ginger are administered simultaneously.
C23. The method as recited in any one of claim C1-C21, wherein the turmeric and the ginger are administered sequentially such that the turmeric and the ginger are simultaneously present in the subject.
D1. A composition of matter comprising ginger and turmeric.
D2. The composition of matter as recited in claim D1, wherein the turmeric is at least 90 wt % curcuminoids selected from the group consisting of curcumin, desmethoxycurcumin, bis-demethoxycurcumin and combinations thereof.
D3. The composition of matter as recited in claim D1, wherein the turmeric is at least 95 wt % curcuminoids selected from the group consisting of curcumin, desmethoxycurcumin, bis-demethoxycurcumin and combinations thereof.
D4. The composition of matter as recited in any one of claims D1-D3, wherein the ginger is ≥20 wt % gingerols and shogaols, 8-10 wt % 6-gingerol, 2-3 wt % 8-gingerol, 3-4 wt % 10-gingerol, 2-3 wt % shogoals with the remainder being ginger polysaccharides, proteins, and native non-volatile components.
D5. The composition of matter as recited in any one of claims D1-D4, wherein the composition consists essentially of the ginger and the turmeric.
D6. The composition of matter as recited in any one of claims D1-D5, wherein the composition provides a synergistic combination index (CI) of 0.8 or less on HeLa cells.
D7. The composition of matter as recited in any one of claims D1-D5, wherein the composition provides a synergistic combination index (CI) of 0.7 or less on HeLa cells.
D8. The composition of matter as recited in any one of claims D1-D5, wherein the composition provides a synergistic combination index (CI) of 0.6 or less on HeLa cells.
D9. The composition of matter as recited in any one of claims D1-D5, wherein the composition provides a synergistic combination index (CI) of 0.5 or less on HeLa cells.
D10. The composition of matter as recited in any one of claims D1-D5, wherein the composition provides a synergistic combination index (CI) of 0.4 or less on HeLa cells.
D11. The composition of matter as recited in any one of claims D1-D5, wherein the composition provides a synergistic combination index (CI) of 0.3 or less on HeLa cells.
D12. The composition of matter as recited in any one of claims D1-D5, wherein the composition provides a synergistic combination index (CI) of 0.2 or less on HeLa cells.
D13. The composition of matter as recited in any one of claims D1-D5, wherein the composition provides a synergistic combination index (CI) of 0.1 or less on HeLa cells.
D14. The composition of matter as recited in any one of claims D1-D13, wherein the composition of matter is formulated as a liquid, foam or a cream.
D15. The composition of matter as recited in any one of claims D1-D13, wherein the composition is incorporated into a vaginal device selected from the group consisting of a tampon, a pessary, a sponge, a male condom and a female condom.
D16. The composition of matter as recited in any one of claims D1-D13, wherein the composition is formulated as a vaginal suppository.
D17. The composition of matter as recited in any one of claims D1-D13, wherein the composition of matter is formulated as tablet.
E1. A method of treating cervical cancer or precancer cells in a subject in need thereof, the method comprising:
   administering to the subject a therapeutically effective amount of a combination of carrageenan and turmeric, wherein the combination provides a synergistic combination index (CI) of 0.8 or less on HeLa cells.
E2. The method as recited claim E1, wherein the combination provides a synergistic combination index (CI) of 0.7 or less.
E3. The method as recited claim E1, wherein the combination provides a synergistic combination index (CI) of 0.6 or less.
E4. The method as recited claim E1, wherein the combination provides a synergistic combination index (CI) of 0.5 or less.
E5. The method as recited claim E1, wherein the combination provides a synergistic combination index (CI) of 0.4 or less.
E6. The method as recited claim E1, wherein the combination provides a synergistic combination index (CI) of 0.3 or less.
E7. The method as recited claim E1, wherein the combination provides a synergistic combination index (CI) of 0.2 or less.
E8. The method as recited claim E1, wherein the combination provides a synergistic combination index (CI) of 0.1 or less.
E9. The method as recited in any one of claims E1-E8, wherein the combination consists essentially of the carrageenan and the turmeric.
E10. The method as recited in any one of claims E1-E9, wherein the synergistic combination index (CI) is measured when the HeLa cells are exposed to a carrageenan concentration of >0 μg/mL to ≤5 μg/mL and a turmeric concentration of >0 μg/mL to ≤2 μg/mL.
E11. The method as recited in any one of claims E1-E9, wherein the synergistic combination index (CI) is measured when the HeLa cells are exposed to a carrageenan concentration of >0 μg/mL to ≤2 g/mL and a turmeric concentration of >0 μg/mL to ≤0.2 μg/mL.
E12. The method as recited in any one of claims E1-E11, wherein the turmeric is at least 90 wt % curcuminoids selected from the group consisting of curcumin, desmethoxycurcumin, bis-demethoxycurcumin and combinations thereof.
E13. The method as recited in any one of claims E1-E11, wherein the turmeric is at least 95 wt % curcuminoids selected from the group consisting of curcumin, desmethoxycurcumin, bis-demethoxycurcumin and combinations thereof.
E14. The method as recited in any one of claims E1-E13, wherein the combination is administered orally.
E15 The method as recited in any one of claims E1-E13, wherein the combination is administered orally and the combination is formulated as a tablet or a liquid.
E16. The method as recited in any one of claims E1-E13, wherein the combination is administered topically to a portion of the subject.
E17. The method as recited in any one of claims E1-E13, wherein the combination is administered topically and the composition is formulated as a liquid, foam or a cream.
E18. The method as recited in any one of claims E1-E13, wherein the combination is administered topically and the combination is formulated as a vaginal suppository.
E19. The method as recited in any one of claims E1-E13, wherein the combination is incorporated into a vaginal device selected from the group consisting of a tampon, a pessary, a sponge, a male condom and a female condom.
E20. The method as recited in any one of claims E16-E19, wherein the therapeutically effective amount is selected to provide a carrageenan concentration of >0 μg/mL to ≤4 μg/mL and a turmeric concentration of >0 μg/mL to ≤4 μg/mL to the portion of the subject.

E21. The method as recited in any one of claim E1-E20, wherein the turmeric and the carrageenan are administered simultaneously.
E22. The method as recited in any one of claim E1-E20, wherein the turmeric and the carrageenan are administered sequentially such that the turmeric and the carrageenan are simultaneously present in the subject.
F1. A composition of matter comprising carrageenan and turmeric.
F2. The composition of matter as recited in claim F1, wherein the turmeric is at least 90 wt % curcuminoids selected from the group consisting of curcumin, desmethoxycurcumin, bis-demethoxycurcumin and combinations thereof.
F3. The composition of matter as recited in claim F1, wherein the turmeric is at least 95 wt % curcuminoids selected from the group consisting of curcumin, desmethoxycurcumin, bis-demethoxycurcumin and combinations thereof.
F4. The composition of matter as recited in any one of claims F1-F3, wherein the composition consists essentially of the carrageenan and the turmeric.
F5. The composition of matter as recited in any one of claims F1-F4, wherein the composition provides a synergistic combination index (CI) of 0.8 or less on HeLa cells.
F6. The composition of matter as recited in any one of claims F1-F4, wherein the composition provides a synergistic combination index (CI) of 0.7 or less on HeLa cells.
F7. The composition of matter as recited in any one of claims F1-F4, wherein the composition provides a synergistic combination index (CI) of 0.6 or less on HeLa cells.
F8. The composition of matter as recited in any one of claims F1-F4, wherein the composition provides a synergistic combination index (CI) of 0.5 or less on HeLa cells.
F9. The composition of matter as recited in any one of claims F1-F4, wherein the composition provides a synergistic combination index (CI) of 0.4 or less on HeLa cells.
F10. The composition of matter as recited in any one of claims F1-F4, wherein the composition provides a synergistic combination index (CI) of 0.3 or less on HeLa cells.

F11. The composition of matter as recited in any one of claims F1-F4, wherein the composition provides a synergistic combination index (CI) of 0.2 or less on HeLa cells.
F12. The composition of matter as recited in any one of claims F1-F4, wherein the composition provides a synergistic combination index (CI) of 0.1 or less on HeLa cells.
F13. The composition of matter as recited in any one of claims F1-F12, wherein the composition of matter is formulated as a liquid, foam or a cream.
F14. The composition of matter as recited in any one of claims F1-F12, wherein the composition is incorporated into a vaginal device selected from the group consisting of a tampon, a pessary, a sponge, a male condom and a female condom.
F15. The composition of matter as recited in any one of claims F1-F12, wherein the composition is formulated as a vaginal suppository.
F16. The composition of matter as recited in any one of claims F1-F12, wherein the composition of matter is formulated as tablet.
G1. A method of treating colon cancer or precancer cells in a subject in need thereof, the method comprising:
   administering to the subject a therapeutically effective amount of a combination of kava and ginger, wherein the combination provides a synergistic combination index (CI) of 0.8 or less on HT29 cells.
G2. The method as recited claim G1, wherein the combination provides a synergistic combination index (CI) of 0.7 or less.
G3. The method as recited claim G1, wherein the combination provides a synergistic combination index (CI) of 0.6 or less.
G4. The method as recited claim G1, wherein the combination provides a synergistic combination index (CI) of 0.5 or less.
G5. The method as recited claim G1, wherein the combination provides a synergistic combination index (CI) of 0.4 or less.
G6. The method as recited claim G1, wherein the combination provides a synergistic combination index (CI) of 0.3 or less.
G7. The method as recited claim G1, wherein the combination provides a synergistic combination index (CI) of 0.2 or less.
G8. The method as recited claim G1, wherein the combination provides a synergistic combination index (CI) of 0.1 or less.
G9. The method as recited in any one of claims G1-G8, wherein the combination consists essentially of the kava and the ginger.
G10. The method as recited in any one of claims G1-G9, wherein the synergistic combination index (CI) is measured when the HT29 cells are exposed to a kava concentration of >0 μg/mL to ≤2 μg/mL and a ginger concentration of >0 μg/mL to ≤20 μg/mL.
G11. The method as recited in any one of claims G1-G9, wherein the synergistic combination index (CI) is measured when the HT29 cells are exposed to a kava concentration of >0 g/mL to ≤1 μg/mL and a ginger concentration of >0 μg/mL to ≤10 μg/mL.
G12. The method as recited in any one of claims G1-G11, wherein the kava is 40-45 wt % dihydrokavain, 15-20 wt % kavain, 15-20 wt % dihydromethysticin, >0 but ≤5 wt % methysticin, 5-10 wt % yangonin and 3-5 wt % demethoxyyangonin with the remainder being native kava components.
G13. The method as recited in any one of claims G1-G12, wherein the ginger is ≥20 wt % gingerols and shogaols, 8-10 wt % 6-gingerol, 2-3 wt % 8-gingerol, 3-4 wt % 10-gingerol, 2-3 wt % shogoals with the remainder being ginger polysaccharides, proteins, and native non-volatile components.
G14. The method as recited in any one of claims G1-G13, wherein the combination is administered orally.
G15 The method as recited in any one of claims G1-G13, wherein the combination is administered orally and the combination is formulated as a tablet or a liquid.
G16. The method as recited in any one of claims G1-G13, wherein the combination is administered topically to a portion of the subject.
G17. The method as recited in any one of claims G1-G13, wherein the combination is administered topically and the combination is formulated as a liquid, foam or a cream.
G18. The method as recited in any one of claims G1-G13, wherein the combination is administered topically and the combination is formulated as an anal suppository.
G19. The method as recited in any one of claims G16-G18, wherein the therapeutically effective amount is selected to provide a kava concentration of >0 g/mL to ≤2 μg/mL and a ginger concentration of >0 g/mL to ≤10 μg/mL to the portion of the subject.
G20. The method as recited in any one of claim G1-G19, wherein the kava and the ginger are administered simultaneously.
G21. The method as recited in any one of claim G1-G19, wherein the kava and the ginger are administered sequentially such that the kava and the ginger are simultaneously present in the subject.
H1. A composition of matter comprising kava and ginger.
H2. The composition of matter as recited in claim H2, wherein the composition consists essentially of the kava and the ginger.
H3. The composition of matter as recited in claim H2, wherein the composition provides a synergistic combination index (CI) of 0.8 or less on HT29 cells.
H4. The composition of matter as recited in claim H2, wherein the composition provides a synergistic combination index (CI) of 0.7 or less on HT29 cells.
H5. The composition of matter as recited in claim H2, wherein the composition provides a synergistic combination index (CI) of 0.6 or less on HT29 cells.
H6. The composition of matter as recited in claim H2, wherein the composition provides a synergistic combination index (CI) of 0.5 or less on HT29 cells.
H7. The composition of matter as recited in claim H2, wherein the composition provides a synergistic combination index (CI) of 0.4 or less on HT29 cells.
H8. The composition of matter as recited in claim H2, wherein the composition provides a synergistic combination index (CI) of 0.3 or less on HT29 cells.
H9. The composition of matter as recited in claim H2, wherein the composition provides a synergistic combination index (CI) of 0.2 or less on HT29 cells.
H10. The composition of matter as recited in claim H2, wherein the composition provides a synergistic combination index (CI) of 0.1 or less on HT29 cells.
H11. The composition of matter as recited in any one of claims H1-H10, wherein the kava is 40-45 wt % dihydrokavain, 15-20 wt % kavain, 15-20 wt % dihydromethysticin, >0 but ≤5 wt % methysticin, 5-10 wt % yangonin and 3-5 wt % demethoxyyangonin with the remainder being native kava components.
H12. The composition of matter as recited in any one of claims H1-H11, wherein the ginger is ≥20 wt % gingerols and shogaols, 8-10 wt % 6-gingerol, 2-3 wt % 8-gingerol, 3-4 wt % 10-gingerol, 2-3 wt % shogoals with the remainder being ginger polysaccharides, proteins, and native non-volatile components.
H13. The composition of matter as recited in any one of claims H1-H12, wherein the composition of matter is formulated as a liquid, foam or a cream.
H14. The composition of matter as recited in any one of claims H1-H12, wherein the composition is formulated as an anal suppository.
H15. The composition of matter as recited in any one of claims H1-H12, wherein the composition of matter is formulated as tablet.
I1. A method of treating colon cancer or precancer cells in a subject in need thereof, the method comprising:
   administering to the subject a therapeutically effective amount of a combination of dihydroxymethysticin (DHM) and turmeric, wherein the composition provides a synergistic combination index (CI) of 0.8 or less on HeLa cells.
I2. The method as recited claim I1, wherein the combination provides a synergistic combination index (CI) of 0.7 or less.
I3. The method as recited claim I1, wherein the combination provides a synergistic combination index (CI) of 0.6 or less.
I4. The method as recited claim I1, wherein the combination provides a synergistic combination index (CI) of 0.5 or less.
I5. The method as recited claim I1, wherein the combination provides a synergistic combination index (CI) of 0.4 or less.
I6. The method as recited claim E1, wherein the combination provides a synergistic combination index (CI) of 0.3 or less.
I7. The method as recited claim I1, wherein the combination provides a synergistic combination index (CI) of 0.2 or less.
I8. The method as recited claim E1, wherein the combination provides a synergistic combination index (CI) of 0.1 or less.
I9. The method as recited in any one of claims I1-I8, wherein the combination consists essentially of the dihydroxymethysticin (DHM) and the turmeric.
I10. The method as recited in any one of claims I1-I9, wherein the synergistic combination index (CI) is measured when the HT29 cells are exposed to a dihydroxymethysticin (DHM) concentration of >0 μg/mL to ≤4 μg/mL and a turmeric concentration of >0 μg/mL to ≤4 μg/mL.
I11. The method as recited in any one of claims I1-I9, wherein the synergistic combination index (CI) is measured when the HT29 cells are exposed to a dihydroxymethysticin (DHM) concentration of >0 g/mL to ≤2 μg/mL and a turmeric concentration of >0 μg/mL to ≤2 μg/mL.
I12. The method as recited in any one of claims I1-I11, wherein the turmeric is at least 90 wt % curcuminoids selected from the group consisting of curcumin, desmethoxycurcumin, bis-demethoxycurcumin and combinations thereof.
I13. The method as recited in any one of claims I1-I11, wherein the turmeric is at least 95 wt % curcuminoids selected from the group consisting of curcumin, desmethoxycurcumin, bis-demethoxycurcumin and combinations thereof.
I14. The method as recited in any one of claims I1-I13, wherein the combination is administered orally.
I15 The method as recited in any one of claims I1-I13, wherein the combination is administered orally and the combination is formulated as a tablet or a liquid.
I16. The method as recited in any one of claims I1-I13, wherein the combination is administered topically to a portion of the subject.
I17. The method as recited in any one of claims I1-I13, wherein the combination is administered topically and the combination is formulated as a liquid, foam or a cream.
I18. The method as recited in any one of claims I1-I13, wherein the combination is administered topically and the combination is formulated as an anal suppository.
I19. The method as recited in any one of claims I16-I18, wherein the therapeutically effective amount is selected to provide a dihydroxymethysticin (DHM) concentration of >0 μg/mL to ≤2 μg/mL and a turmeric concentration of >0 μg/mL to ≤3 μg/mL to the portion of the subject.
I20. The method as recited in any one of claim I1-I19, wherein the turmeric and the dihydroxymethysticin (DHM) are administered simultaneously.
I21. The method as recited in any one of claim I1-I19, wherein the turmeric and the dihydroxymethysticin (DHM) are administered sequentially such that the turmeric and the dihydroxymethysticin (DHM) are simultaneously present in the subject.
J1. A composition of matter comprising dihydroxymethysticin (DHM) and turmeric.
J2. The composition of matter as recited in claim J1, wherein the turmeric is at least 90 wt % curcuminoids selected from the group consisting of curcumin, desmethoxycurcumin, bis-demethoxycurcumin and combinations thereof.
J3. The composition of matter as recited in claim J1, wherein the turmeric is at least 95 wt % curcuminoids selected from the group consisting of curcumin, desmethoxycurcumin, bis-demethoxycurcumin and combinations thereof.
J4. The composition of matter as recited in any one of claims J1-J3, wherein the composition consists essentially of the dihydroxymethysticin (DHM) and the turmeric.
J5. The composition of matter as recited in any one of claims J1-J4, wherein the composition provides a synergistic combination index (CI) of 0.8 or less on HT29 cells.
J6. The composition of matter as recited in any one of claims J1-J4, wherein the composition provides a synergistic combination index (CI) of 0.7 or less on HT29 cells.
J7. The composition of matter as recited in any one of claims J1-J4, wherein the composition provides a synergistic combination index (CI) of 0.6 or less on HT29 cells.
J8 The composition of matter as recited in any one of claims J1-J4, wherein the composition provides a synergistic combination index (CI) of 0.5 or less on HT29 cells.
J9. The composition of matter as recited in any one of claims J1-J4, wherein the composition provides a synergistic combination index (CI) of 0.4 or less on HT29 cells.
J10. The composition of matter as recited in any one of claims J1-J4, wherein the composition provides a synergistic combination index (CI) of 0.3 or less on HT29 cells.
J11. The composition of matter as recited in any one of claims J1-J4, wherein the composition provides a synergistic combination index (CI) of 0.2 or less on HT29 cells.
J12. The composition of matter as recited in any one of claims J1-J4, wherein the composition provides a synergistic combination index (CI) of 0.1 or less on HT29 cells.
J13. The composition of matter as recited in any one of claims J1-J12, wherein the composition of matter is formulated as a liquid, foam or a cream.
J14. The composition of matter as recited in any one of claims J1-J12, wherein the composition is formulated as an anal suppository.
J15. The composition of matter as recited in any one of claims J1-J12, wherein the composition of matter is formulated as tablet.
K1. A method of treating colon cancer or precancer cells in a subject in need thereof, the method comprising:
   administering to the subject a therapeutically effective amount of a combination of dihydroxymethysticin (DHM) and 5-fluorouracil (5-FU), wherein the composition provides a synergistic combination index (CI) of 0.8 or less on HT29 cells.
K2. The method as recited claim K1, wherein the combination provides a synergistic combination index (CI) of 0.7 or less.
K3. The method as recited claim K1, wherein the combination provides a synergistic combination index (CI) of 0.6 or less.
K4. The method as recited claim K1, wherein the combination provides a synergistic combination index (CI) of 0.5 or less.
K5. The method as recited claim K1, wherein the combination provides a synergistic combination index (CI) of 0.4 or less.
K6. The method as recited claim K1, wherein the combination provides a synergistic combination index (CI) of 0.3 or less.
K7. The method as recited claim K1, wherein the combination provides a synergistic combination index (CI) of 0.2 or less.
K8. The method as recited claim K1, wherein the combination provides a synergistic combination index (CI) of 0.1 or less.
K9. The method as recited in any one of claims K1-K8, wherein the combination consists essentially of the dihydroxymethysticin (DHM) and the 5-fluorouracil (5-FU).
K10. The method as recited in any one of claims K1-K9, wherein the synergistic combination index (CI) is measured when the HT29 cells are exposed to a dihydroxymethysticin (DHM) concentration of >0 μg/mL to ≤10 μg/mL and a 5-fluorouracil (5-FU) concentration of >0 μg/mL to ≤5 μg/mL.
K11. The method as recited in any one of claims K1-K9, wherein the synergistic combination index (CI) is measured when the HT29 cells are exposed to a dihydroxymethysticin (DHM) concentration of >0 μg/mL to ≤5 μg/mL and a 5-fluorouracil (5-FU) concentration of >0 μg/mL to ≤2 μg/mL.
K12. The method as recited in any one of claims K1-K9, wherein the synergistic combination index (CI) is measured when the HT29 cells are exposed to a dihydroxymethysticin (DHM) concentration of >0 μg/mL to ≤1 μg/mL and a 5-fluorouracil (5-FU) concentration of >0 μg/mL to ≤1 μg/mL.
K13. The method as recited in any one of claims K1-K9, wherein the synergistic combination index (CI) is measured when the HT29 cells are exposed to a dihydroxymethysticin (DHM) concentration of >0 μg/mL to ≤1 μg/mL and a 5-fluorouracil (5-FU) concentration of >0 μg/mL to ≤1 μg/mL.
K14. The method as recited in any one of claims K1-K13, wherein the combination is administered orally.
K15 The method as recited in any one of claims K1-K13, wherein the combination is administered orally and the combination is formulated as a tablet or a liquid.
K16. The method as recited in any one of claims K1-K13, wherein the combination is administered topically to a portion of the subject.
K17. The method as recited in any one of claims K1-K13, wherein the combination is administered topically and the combination is formulated as a liquid, foam or a cream.
K18. The method as recited in any one of claims K1-K13, wherein the combination is administered topically and the combination is formulated as an anal suppository.
K19. The method as recited in any one of claims K16-K18, wherein the therapeutically effective amount is selected to provide a dihydroxymethysticin (DHM) concentration of >0 g/mL to ≤2 μg/mL and a 5-fluorouracil (5-FU) concentration of >0 μg/mL to ≤10 μg/mL to the portion of the subject.
K20. The method as recited in any one of claim K1-K19, wherein the 5-fluorouracil (5-FU) and the dihydroxymethysticin (DHM) are administered simultaneously.
K21. The method as recited in any one of claim K1-K19, wherein the 5-fluorouracil (5-FU) and the dihydroxymethysticin (DHM) are administered sequentially such that the 5-fluorouracil (5-FU) and the dihydroxymethysticin (DHM) are simultaneously present in the subject.
L1. A composition of matter comprising dihydroxymethysticin (DHM) and 5-fluorouracil (5-FU).
L2. The composition of matter as recited in claim L2, wherein the composition consists essentially of the dihydroxymethysticin (DHM) and the 5-fluorouracil (5-FU).
L3. The composition of matter as recited in claim L2, wherein the composition provides a synergistic combination index (CI) of 0.8 or less on HT29 cells.
L4. The composition of matter as recited in claim L2, wherein the composition provides a synergistic combination index (CI) of 0.7 or less on HT29 cells.
L5. The composition of matter as recited in claim L2, wherein the composition provides a synergistic combination index (CI) of 0.6 or less on HT29 cells.
L6. The composition of matter as recited in claim L2, wherein the composition provides a synergistic combination index (CI) of 0.5 or less on HT29 cells.
L7. The composition of matter as recited in claim L2, wherein the composition provides a synergistic combination index (CI) of 0.4 or less on HT29 cells.
L8. The composition of matter as recited in claim L2, wherein the composition provides a synergistic combination index (CI) of 0.3 or less on HT29 cells.
L9. The composition of matter as recited in claim L2, wherein the composition provides a synergistic combination index (CI) of 0.2 or less on HT29 cells.
L10. The composition of matter as recited in claim L2, wherein the composition provides a synergistic combination index (CI) of 0.1 or less on HT29 cells.
L11. The composition of matter as recited in any one of claims L1-L10, wherein the composition of matter is formulated as a liquid, foam or a cream.
L12. The composition of matter as recited in any one of claims L1-L10, wherein the composition is formulated as an anal suppository.
L13. The composition of matter as recited in any one of claims L1-L10, wherein the composition of matter is formulated as tablet.

What is claimed is:

1. A composition of matter comprising (1) turmeric and tanshinone IIA wherein the composition of matter is free of both epigallocatechin-3-gallate (EGCG) and resveratrol, (2) turmeric and ginger or (3) turmeric and carrageenan.

2. The composition of matter as recited in claim 1, wherein the composition of matter consists essentially of the tanshinone IIA and the turmeric.

3. The composition of matter as recited in claim 1, wherein the composition of matter comprises turmeric and tanshinone IIA, wherein the turmeric is at least 90 wt % curcuminoids selected from the group consisting of curcumin. desmethoxycurcumin, bis-demethoxycurcumin and combinations thereof.

4. The composition of matter as recited in claim 1, wherein the composition of matter comprises turmeric and tanshinone IIA and the composition of matter is formulated as a liquid, foam or a cream.

5. The composition of matter as recited in claim 1, wherein the composition of matter comprises turmeric and tanshinone IIA and the composition of matter is incorporated into a vaginal device selected from the group consisting of a tampon, a pessary, a sponge, a male condom and a female condom.

6. The composition of matter as recited in claim 2, wherein the composition of matter provides a synergistic combination index (CI) of 0.8 or less on HeLa cells.

7. The composition of matter as recited in claim 2, wherein the composition of matter is incorporated into a vaginal device selected from the group consisting of a tampon, a pessary, a sponge, a male condom and a female condom.

8. The composition of matter as recited in claim 2, wherein the composition of matter is formulated as a liquid, foam, vaginal suppository or a cream.

9. A method of treating cervical cancer or precancer cells in a subject in need thereof, the method comprising:
administering to the subject a therapeutically effective amount of a combination of therapeutics selected from the group consisting of (1) turmeric and tanshinone IIA, (2) turmeric and ginger and (3) turmeric and carrageenan, wherein the combination of therapeutics provides a synergistic combination index (CI) of 0.8 or less on HeLa cells.

10. The method as recited in claim 9, wherein the combination of therapeutics is turmeric and tanshinone IIA.

11. The method as recited in claim 10, wherein the combination of therapeutics is free of both epigallocatechin-3-gallate (EGCG) and resveratrol.

12. The method as recited in claim 11, wherein the turmeric is at least 90 wt % curcuminoids selected from the group consisting of curcumin, desmethoxycurcumin, bis-demethoxycurcumin and combinations thereof.

13. The method as recited in claim 11, wherein the combination of therapeutics is administered orally.

14. The method as recited in claim 11, wherein the combination of therapeutics is administered topically to a portion of the subject.

15. The method as recited in claim 14, wherein the therapeutically effective amount is selected to provide a tanshinone IIA concentration of >0 μM to ≤5 μM and a turmeric concentration of >0 μg/mL to ≤4 μg/mL to the portion of the subject.

16. The method as recited in claim 11, wherein the combination of therapeutics is administered topically and the combination of therapeutics is formulated as a liquid, foam or a cream such that the administrating of the combination of therapeutics occurs simultaneously.

17. The method as recited in claim 11, wherein the combination of therapeutics is administered topically and the combination of therapeutics is formulated as a vaginal suppository such that the administrating of the combination of therapeutics occurs simultaneously.

18. The method as recited in claim 11, wherein the combination of therapeutics is incorporated into a vaginal device selected from the group consisting of a tampon, a pessary, a sponge, a male condom and a female condom such that the administrating of the combination of therapeutics occurs simultaneously.

19. The method as recited in claim 10, wherein the combination of therapeutics consists essentially of the turmeric and the tanshinone IIA and the administering occurs simultaneously.

20. A method of treating colon cancer or precancer cells in a subject in need thereof, the method comprising:
administering to the subject a therapeutically effective amount of a combination of therapeutics selected from the group consisting of (1) kava and ginger, (2) dihydroxymethysticin (DHM) and turmeric and (3) dihydroxymethysticin (DHM) and 5-fluorouracil (5-FU), wherein the combination of therapeutics provides a synergistic combination index (CI) of 0.8 or less on HT29 cells.
